(19) United States
(12) Patent Application Publication
(10) Pub. No.: US 2004/0016015 A1
(43) Pub. Date: Jan. 22, 2004

(54) High efficiency germline transformation system

(76) Inventors: Thanh-Tuyen T. Nguyen, Raleigh, NC (US); George C. Allen, Raleigh, NC (US); Georgia L. Helmer, Alexandria, VA (US); William F. Thompson, Raleigh, NC (US)

Correspondence Address: MYERS BIGEL SIBLEY & SAJOVEC, PO BOX 37428, RALEIGH, NC, 27627, US

(21) Appl. No.: 10/447,032

(22) Filed: May 28, 2003

Related U.S. Application Data
(63) Continuation-in-part of application No. 10/196,771, filed on Jul. 17, 2002.

(51) Int. Cl. A01H 1/00; C12N 15/82; A01H 5/00

(52) U.S. Cl. 800/278; 800/320.1; 800/320; 800/320.2; 800/320.3

(57) ABSTRACT

A method for introducing a heterologous nucleic acid of interest into a plant to thereby produce a recombinant plant is carried out by (a) providing a recombinant nucleic acid of interest, the recombinant nucleic acid comprising the heterologous nucleic acid of interest, and preferably including (i) a matrix attachment region (MAR) positioned 5′ to the heterologous DNA, (ii) a MAR positioned 3′ to the heterologous nucleic acid of interest, or (iii) a MAR positioned 5′ to the heterologous nucleic acid of interest and a MAR positioned 3′ to the heterologous nucleic acid of interest; (b) providing meristem tissue of the plant of interest; (c) introducing the recombinant nucleic acid of interest into the meristem tissue; and then (d) propagating a recombinant plant from the meristem tissue, preferably by a direct propagation technique.

### RELATED APPLICATIONS

This application is a continuation-in-part of commonly owned, copending application Ser. No. 10/196,771, filed Jul. 17, 2002, the disclosure of which is incorporated by reference herein in its entirety.

### FIELD OF THE INVENTION

The present invention concerns methods of introducing heterologous nucleic acids into plants such as maize.

### BACKGROUND OF THE INVENTION

Maize and other plants are modified to incorporate foreign, or “heterologous”, DNA by a variety of means. Available techniques include direct DNA delivery techniques such as ballistic bombardment and electroporation, and delivery through biological vectors such as Agrobacterium or viruses.

A problem with DNA transformation techniques is the need for intermediate tissue culturing steps between transformation of cells with the vector of choice and subsequent plant propagation. In general, cells of plants are transformed, the transformed cells cultured and selected, a first generation plant is regenerated from the cultured cells, and subsequent generations of plants are propagated from the first generation plants. However, some plants are not amenable to tissue culture. Hence, while plant cells can be transformed to incorporate heterologous DNA, intact plants cannot be generated from the transformed cells.

For those plants that are amenable to tissue culture, somaclonal variation may be a problem. Somaclonal variation is the hereditable variation found among somatic clones of the same plant which occurs during tissue culturing of cells derived from that plant. When attempting to introduce introduce new genetic material into elite lines of plants, somoclonial variation can lead to a degradation of the elite phenotype which made those plants desirable targets for transformation in the first place.

Finally, tissue culturing is a relatively time consuming and expensive step in the plant transformation process. Accordingly, there is a need for new ways to transform plant species such as maize without the need for an intervening tissue culturing step.

### SUMMARY OF THE INVENTION

A method for introducing a heterologous nucleic acid of interest into a plant to thereby produce a recombinant plant is disclosed. In general, the method comprising the steps of:

(a) providing a recombinant nucleic acid of interest, the recombinant nucleic acid comprising the heterologous nucleic acid (e.g., DNA) of interest;

(b) providing meristem tissue of the plant of interest;

(c) introducing the recombinant nucleic acid of interest into the meristem tissue; and then

(d) propagating a recombinant plant from the meristem tissue, preferably by a direct propagation technique.

In one embodiment of the invention the recombinant nucleic acid comprises the heterologous nucleic acid (e.g., DNA) of interest, and further includes (i) a matrix attachment region (MAR) positioned 5′ to the heterologous nucleic acid of interest, (ii) a MAR positioned 3′ to the heterologeous nucleic acid of interest, or (iii) a MAR positioned 5′ to the heterologous nucleic acid of interest and a MAR positioned 3′ to the heterologous nucleic acid of interest.

In a preferred embodiment of the invention, the introducing step is carried out in a manner that introduces the recombinant nucleic acid of interest into the meristem L2 layer.

In a preferred embodiment of the invention, recombinant plants produced and propagated in accordance with the invention comprise, in the mature plant, gametophyte cells that carry or contain the recombinant nucleic acid of interest. Such gametophyte cells may be malle gametophyte cells (e.g., anther cells) or female gametophyte cells.

The plant is generally a vascular plant and may be of any suitable type, including dicots and monocots. Grass species such as maize, wheat, oats, rye, barley, sorghum, and rice are preferred. The plant may be a hybrid plant or an inbred plant.

The introducing step may be carried out by any suitable technique, including but not limited to direct nucleic acid/DNA delivery (e.g., microparticle bombardment) and Agrobacterium-mediated transformation.

The method may further comprise the step of sexually propagating the plant to produce a plant that is hemizygous or homozygous for the heterologous nucleic acid/DNA of interest.

In one embodiment, the heterologous nucleic acid of interest comprises a structural gene operably associated with a promoter active in cells of the plant, and cells of the plant exhibit increased expression of the structural gene as compared to cells of the same plant that do not contain the heterologous nucleic acid of interest.

Plants produced by the foregoing processes, as well as pollen, seed and other propagules thereof, crops comprised of a plurality of such plants planted together in a common agricultural field, along with plant portions taken from such plants such as shoots, roots, tubers, fruits, and vegetables, are also aspects of the present invention.

The foregoing and other objects and aspects of the present invention are explained in greater detail below.

### BRIEF DESCRIPTION OF THE DRAWINGS

FIG. 1: SEM image of a developing embryo at the stage to be bombarded (A. Van Lammereen, Acta Bot Neerl. 35, 169-188 (1986)).

FIG. 2: GFP fluorescence of transformed embryos (C, D) compared with untransformed (A) and bombarded (B) controls. SM, shoot meristem.

FIG. 3: Plants derived from bombarded embryos and grown to maturity in the Phytotron.

FIG. 4: Representative PCR results for 6 plants (lanes 2-8) plus positive (lane 1) and negative (lane 9) controls. Amplifications used mas sense and gfp antisense primers.

FIG. 5: Expression of GUS transgene in a double MAR construct bombarded into 11 DAP

embryos of the inbred line M37W.

FIG. 5A: Transient expression of GUS on the embryonic axis side of an immature embryo.

FIG. 5B: A transgenic sector on a leaf of a To plantlet.

FIG. 5C: GUS sectors on an inflorescence part histochemically assayed before tassel emergence.

FIG. 5D: A GUS-expressing floret with blue stained floral whorl and anthers.

FIG. 5E: Chimeric anthers inside florets.

FIG. 5F: GUS-expressing anthers vs non-transgenic anthers.

FIG. 5G: Close-up picture of young anthers expressing GUS assayed before emergence of tassels.

### DETAILED DESCRIPTION OF THE PREFERRED EMBODIMENTS

In general, the technology described herein involves, in various embodiments, germline transformation by introducing and expressing nucleic acid or DNA of interest in meristematic cells/cell layers, of shoot meristem of immature embryos, mature seeds, seedlings or plants, that give rise to transformed sectors including reproductive tissues/cells Such as pollen and egg cells. The chimeric, transgenic plants are recovered from germination of immature embryos and seeds or by clonal propagation of lateral buds, therefore avoiding the problem of somaclonal variation and preserving the genetic background of mother plants before introduction of DNA of interest. Increase in transformation efficiency is achieved by using the Matrix Attachment Regions (MAR) flanking the DNA cassette and screenable reporter gene to avoid drug selection of primary tranformant and shortening the time required to produce transgenic plants.

Nucleotide sequences are presented herein by single strand only, in the 5′ to 3′ direction, from left to right.

Applicants specifically intend that the disclosures of all U.S. patent references cited herein are to be incorporated herein by reference.

1. General Definitions.

“Nucleic acid” herein refers to any type of nucleic acid, including DNA and RNA.

“Plant” as used herein refers to vascular plants, including both angiosperms and gymnosperms and both monocots and dicots.

“Inbred” plant as used herein refers to a plant or plant line that has been repeatedly crossed or inbred to achieve a high degree of genetic uniformity, and low heterozygosity, as is known in the art.

“Hybrid” plant as used herein refers to a plant that is the product of a cross between two genetically different parental plants, as is known in the art.

“Germline transformation” refers to introducing and stably expressing DNA of choice in meristem cells/cell layers that give rise to transformed reproductive tissues and gametes, e.g. pollen, egg cells.

“Meristem” refers to a plant structure composed of a localized group of actively dividing cells, from which permanent tissue system (root, shoot, leaf, flower) are derived. The main categories of meristems are: apical meristems (in root and shoot tips), lateral meristems (vascular and cork cambiums) and inter-callary meristems (in the nodal region and at the base of certain leaves). In this patent, the term meristems refers to both shoot apical meristems that produces main shoots and axillary meristems that give rise to axillary buds/branches.

“In vitro technique(s)” refers to techniques that involve growing embryos, organs, tissues or cells that are detached from “mother” plants, in a nutrient medium under aseptic environment to allow complete plant development, perpetual growth or regeneration of whole plants.

“In vitro germination” refers to a natural course of development encompassing stages from zygote to complete plant in a nutrient medium under aseptic environment provided to an embryo that is removed from the ovule (ex-ovular).

“Tissue culture” refers to a process of growing cells, tissues or organs in a nutrient medium under aseptic condition to allow perpetual growth and/or multiplication of plants either by forming adventitious structures (e.g. shoots, roots) or regenerating plants from callus that derives from disorganized proliferation of cells.

“Directly propagating” as used herein refers to the propogation of a plant (e.g., a structure having at least shoots, and preferably stems and leaves) from tissue (preferably apical meristem tissue) into which a heterologous nucleic acid of interest has been introduced, without an intervening chemical selection step, and without an intervening regeneration step or tissue culture step. Optionally but preferably the direct propagating step serves to reduce the occurrence of somaclonal variation.

“Regeneration” refers to a process in tissue culture involving a morphogenetic response that results in the production of new organs, somatic embryos or whole plants from cultured explants or calli derived from them. The term “regeneration” herein includes the process of shoot multiplication as described in Lowe et al., Biotechnology 13, 677-682 (1995).

“Somaclonal variation” refers to heritable differences among plants propagated through tissue culture of a single mother plant.

“Drug selection” refers to exposure of plant material to antibiotics or other drugs with the intent to kill or inhibit the growth of non-transformed cells lacking an appropriate gene to resist the effects of the drug.

“Screenable marker” refers to a gene that, when present and expressed in a plant or plant cell, causes a phenotype that can be detected as an indication of transformation. Examples include, but are not limited to GUS, GFP, and genes encoding anthocyanin pigments.

“Operatively associated,” as used herein, refers to DNA sequences on a single DNA molecule which are associated so that the function of one is affected by the other. Thus, a transcription initiation region is operatively associated with a structural gene when it is capable of affecting the expression of that structural gene (i.e., the structural gene is under the transcriptional control of the transcription initiation region). The transcription initiation region is said to be “upstream” from the structural gene, which is in turn said to be “downstream” from the transcription initiation region.

2. Matrix Attachment Regions.

MARs (also called scaffold attachment regions, or “SARs”) that are used to carry out the present invention may be of any suitable origin. In general, the MAR of any eukaryotic organism (including plants, animals, and yeast) may be employed, as Mars are highly conserved among the eukaryotes. See, e.g., G. Allen et al., The Plant Cell 5, 603-613 (1993); M. Eva Luderus et al., Cell 70, 949-959 (1992); G. Hall et al., Proc. Natl. Acad. Sci. USA 88, 9320-9324 (1991). For example, animal MARs are shown to be operational in plants in P. Breyve, The Plant Cell 4, 463-471 (1992), and yeast MARs are shown to be operational in plants hereinbelow. Plant MARs may be taken from any suitable plant, including those plants specified above and below; animal MARs may be taken from any suitable animal including mammals (e.g., dog, cat), birds (e.g., chicken, turkey), etc.; and MARs may be taken from other eukaryotes such as fungi (e.g., Saccharomyces cereviseae). Where two matrix attachment regions are employed, they may be the same or different, and may be in the same orientation or opposite orientation. The length of the MAR is not critical so long as it retains operability as an SAR, with lengths of from 400 to 1000 base pairs being typical.

Examples of MARs that may be used to carry out the present invention include, but are not limited to, those described in U.S. Pat. Nos. 5,773,695 and 5,773,689, and in PCT Application WO99/07866 to S. Michalowski and S. Spiker.

3. Plants for Transformation.

Plants which may be employed in practicing the present invention include (but are not limited to) both angiosperms and gymnosperms and monocots and dicots. Particular examples include but are not limited to tobacco (Nicotiana tabacum), potato (Solanum tuberosum), soybean (glycine max), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinesis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amugdalus), sugar beets (Beta vulgaris), corn (Zea mays, also known as maize), wheat, oats, rye, barley, rice, vegetables, ornamentals, and conifers. Vegetables include tomatoes (Lycopersicon esculentum), lettuce (e.g., Lactuea sativa), green beans (Phaseolus vulgaris), lima beans (Phaseolus limensis), peas (Pisum spp.) and members of the genus Cucumis such as cucumber (C. sativus), cantaloupe (C. cantalapensis), and musk melon (C. melo). Ornamentals include azalea (Rhododendron spp.), hydrangea (Macrophylla hydrangea), hibiscus (Hibiscus rosasanesis), roses (Rosa spp.), tulips (Tulipa spp.), daffodils (Narcissus spp.), petunias (Petunia hybrida), carnation (dianthus caryophyllus), poinsettia (Euphorbia pulcherima), and chrysanthemum. Gymnosperms which may be employed to carrying out the present invention include conifers, including pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Particularly preferred for carrying out the present invention are monocots, and more particularly grass species such as wheat, oats, rye, barley, sorghum, rice, and maize. Maize (or corn) is particularly preferred.

4. Recombinant Nucleic Acid Constructs and Transformation Methods.

DNA constructs which are “expression cassettes” used to carry out the present invention preferably include, 5′ to 3′ in the direction of transcription, a transcription initiation region, a structural gene positioned downstream from the transcription initiation region and operatively associated therewith, and at least one MAR positioned upstream and/or downstream thereof, as described above. The promoter should be capable of operating in the cells to be transformed (either constitutively or on a tissue-specific or other inducible basis). A terination region may be provided downstream of the structural gene, which termination region may be derived from the same gene as the promoter region, or may be derived from a different gene.

The transcription initiation region, which includes the RNA polymerase binding site (promoter), may be native to the host plant to be transformed or may be derived from an alternative source, where the region is functional in the host plant. Other sources include the Agrobacterium T-DNA genes, such as the transcriptional initiation regions for the biosynthesis of nopalinie, octapine, manniiopine, or other opine transcriptional initiation regions; transcriptional initiation regions prom plants, such as the ubiquitin promoter, root specific promoters (see, e.g., U.S. Pat. No. 5,459,292 to Conkling et al.; WO 91/13992 to Advanced Technologies); transcriptional initiation regions from viruses (including host specific viruses), or partially or wholly synthetic transcription initiation regions. Transcriptional initiation and termination regions are well known (see, e.g., dGreve, J. Mol. Appl. Genet. 1, 499-511 (1983); Salomon et al., EMBO J. 3, 141-146 (1984); Garfinkel et al., Cell 27, 143-153 (1983); Barker et al., Plant Mol. Biol. 2, 235-350 (1983)); including various promoters isolated from plants (see, e.g., U.S. Pat. No. 4,962,028) and viruses (such as the cauliflower mosaic virus promoter, CaMV 35S).

The transcriptional initiation regions may, in addition to the RNA polymerase binding site, include regions which regulate transcription, where the regulation involves, for example, chemical or physical repression or induction (e.g., regulation based on metabolites, light, or other physicochemical factors; see, e.g., WO 93/06710 disclosing a nematode responsive promoter) or regulation based on cell differentiation (such as associated with leaves, roots, seed, or the like in plants; see, e.g., U.S. Pat. No. 5,459,252 disclosing a root-specific promoter). Thus, the transcriptional initiation region, or the regulatory portion of such region, is obtained from an appropriate gene which is so regulated. For example, the 1,5-ribulose biphosphate carboxylase gene is light-induced and may be used for transcriptional initiation. Other genes are known which are induced by stress, temperature, wounding, pathogen effects, etc.

The term “structural gene” herein refers to those portions of genes which comprise a DNA segment coding for a protein, polypeptide, or portion thereof, possibly including a ribosome binding site and/or a translational start codon, but lacking a transcription initiation region. The term can also refer to copies of a structural gene naturally found within a cell but artificially introduced. The structural gene may encode a protein not normally found in the plant cell in which the gene is introduced or in combination with the transcription initiation region to which it is operationally associated, in which case it is termed a heterologous structural gene. Genes which may be operationally associated with a transcription initiation region of the present invention for expression in a plant species may be derived from a chromosomal gene, cDNA, a synthetic gene, or combinations thereof. Any structural gene may be employed. The structural gene may encode an enzyme to introduce a desired trait into the plant, such as glyphosphate resistance; the structural gene may encode a protein such as a Bacillus thuringiensis protein (or fragment thereof) to impart insect resistance to the plant; the structural gene may encode a plant virus protein or fragment thereof to impart virus resistance to the plant.

The term “structural gene” as used herein is also intended to encompass a DNA encoding an antisense agent that will bind to a particular mRNA in the plant cell and downregulate translation thereof. See, e.g., U.S. Pat. No. 4,107,065 to Shewmaker et al. A sense construct or agent that will downregulate the expression of a corresponding gene in the plant (e.g., by a mechanism such as “gene silencing”) is also a “structural gene” as used herein.

Expression cassettes useful in methods of the present invention may be provided in a DNA construct which also has at least one replication system. For convenience, it is common to have a replication system functional in Escherichia coli, Such as ColEl, pSCO101, pACYC184, or the like. In this manner, at each stage after each manipulation, the resulting construct may be cloned, sequenced, and the correctness of the manipulation determined. In addition, or in place of the E. coli replication system, a broad host range replication system may be employed, Such as the replication systems of the P-I incompatibility plasmids, e.g., pRK290. In addition to the replication system, there will frequently be at least one marker present, which may be useful in one or more hosts, or different markers for individual hosts. That is, one marker may be employed for selection in a prokaryotic host, while another marker may be employed for selection in a eukaryotic host, particularly a plant host. The markers may be protection against a biocide, such as antibiotics, toxins, heavy metals, or the like; provide complementation, for example by imparting prototrophy to an auxotroplic host; or provide a visible phenotype through the production of a novel compound. Exemplary genes which may be employed include neomycin phosphotransferase (NPTII), hygromycin phosphotranisferase (HPT), chloramphenicol acetyltransferase (CAT), nitrilase, and the gentamicil resistance gene. For plant host selection, non-limiting examples of suitable markers are β-glucuronidase, providing indigo production; luciferase, providing visible light production; NPTII, providing kanamycin resistance or G418 resistance; HPT, providing hygromycin resistance; and the mutated aroA gene, providing glyphosate resistance.

The various fragments comprising the various constructs, expression cassettes, markers, and the like may be introduced consecutively by restriction enzyme cleavage of an appropriate replication system, and insertion of the particular construct or fragment into the available site. After ligation and cloning the DNA construct may be isolated for further manipulation. All of these techniques are amply exemplified in the literature and find particular exemplification in Sambrook et al., Molecular Cloning: A Laboratory Manual, (2d Ed. 1989) (Cold Spring Harbor Laboratory, Cold Spring Harbor, N.Y.).

As used herein, a transgenic plant refers to a plant in which at least some cells are stably transformed with a heterologous DNA construct. As used herein, a heterologous DNA construct refers to DNA which is artificially introduced into a cell or into a cell's ancestor. Such DNA may contain genes or DNA which would not normally be found in the cell to be transformned, or may contain genes or DNA which is contained in the cell to be transformed. In the latter case, cells are transformed so that they contain additional or multiple copies of the DNA sequence or gene of interest.

Vectors which may be used to transform plant tissue with DNA constructs of the present invention include Agrobacterium vectors, direct DNA delivery vectors (particularly ballistic vectors), as well as other known vectors suitable for DNA-mediated transformation such as viral vectors (including viral vectors such as tomato golden mosaic virus, tobacco mosaic virus, and others).

Microparticles carrying a DNA construct of the present invention, which microparticles are suitable for the ballistic transformation of a cell, are useful for transforming cells according to the present invention. The microparticle is propelled into a cell to produce a transformed cell. Where the transformed cell is a plant cell, a plant may be regenerated from the transformed cell according to techniques known in the art. Any suitable ballistic cell transformation methodology and apparatus can be used in practicing the present invention. Exemplary apparatus and procedures are disclosed in Stomp et al., U.S. Pat. No. 5,122,466; and Sanford and Wolf, U.S. Pat. No. 4,945,050 (the disclosures of all U.S. patent references cited herein are incorporated herein by reference in their entirety). When using ballistic transformation procedures, the expression cassette may be incorporated into a plasmid capable of replicating in the cell to be transformed. Examples of microparticles suitable for use in such systems include 1 to 5 μm gold spheres. The DNA construct may be deposited on the microparticle by any suitable technique, such as by precipitation. Such ballistic transformation techniques are useful for introducing foreign genes into a variety of plant species, and are particularly useful for the transformation of monocots.

Vectors that may be used to carry out the present invention include Agrobacterium vectors. Numerous Agrobacterium vectors are known. See, e.g., U.S. Pat. No. 4,536,475 to Anderson; U.S. Pat. No. 4,693,977 to Schliperoort et al.; U.S. Pat. No. 4,886,937 to Sederoff et al.; U.S. Pat. No. 5,501,967 To Offringa et al.; T. Hall et al., EPO Application No. 0122791; R. Fraley et al., Proc. Natl. Acad. Sci. USA 84:4803 (1983); L. Herrera-Estrella et al., EMBO J. 2:987 (1983); G. Helmer et al., bio/Technology 2:520 (1984); N. Murai et al., Science 222:476 (1983). In general, such vectors comprise an agrobacteria, typically Agrobacterium tumefaciens, that carried at least one tumor-inducing (or “Ti”) plasmid. When the agrobacteria is Agrobacterium rhizogenes, this plasmid is also known as the root-inducing (or “Ri”) plasmid. The Ti (or Ri) plasmid contains DNA referred to as “T-DNA” that is transferred to the cells of a host plant when that plant is infected by the agrobacteria. In an Agrobacterium vector, the T-DNA is modified by genetic engineering techniques to contain the “expression cassette”, or the gene or genes of interest to be expressed in the transformned plant cells, along with the associated regulatory sequences. The agrobacteria may contain multiple plasmids, as in the case of a “binary” vector system. Such Agrobacterium vectors are useful for introducing foreign genes into a variety of plant species, and are particularly useful for the transformation of dicots.

The combined use of Agrobacterium vectors and microprojectile bombardment is also known in the art (see, e.g. European Patent Nos. 486233 and 486234 to D. Bidney).

5. Meristems and Plant Propagation.

The meristem may be of any suitable type, including post-germination plant meristem, adult plant meristem, and seedling meristem.

As noted above, propogation of the meristem may be carried out by any suitable technique as long as it is a direct propagation step, without intervening chemical selection or regeneration.

When the meristem comprises embryo meristem, the propagating step may be carried out by in vitro germination. For example, when the plant is a grass species, the meristem tissue may comprise an embryo apical meristem taken at a time of development between the formation of the apical meristem and before the apical meristem is occluded by coleoptile tissue, and the propagating step may be carried out by in vitro germination. More particularly, when the plant is a maize plant, the meristem tissue comprises embryo apical meristem taken at a time of development from 7 to 14 days after pollination, and the propagating step may be carried out by in vitro germination.

In vitro embryogenesis is known and may be carried out in accordance with known techniques, or variations thereof which will be apparent to those skilled in the art based upon the instant disclosure. The first attempts with immature embryos were made by Hannig in 1904 (REF: Hannig, E. 1904. Zur physiologic pflanzlicher embryonen. Ueber die kultur von Cruciferen-Embryonen ausserhalb des embryosacks. Bot. Zeit.,62, 45). He successfully grew cruciferous embryos on a simple medium. He emphasized the importance of a high concentration of sugar to prevent the embryo from germinating before maturity. We used 12% sucrose in our embryo maturation medium herein.

A more recent review on “Culture of Zygotic Embryos” was written by Michel Monnier in T. A. Thorpe (ed), In Vitro Embryogenesis in Plants, pp. 117-153 (1995).

Meristem proliferation is a technique in which meristems are excised from the plant and propagated in organ culture to produce more meristems. However, such techniques do not include a differentiation step and the meristems are always multicellular entities, which makes such techniques different from regeneration protocols.

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, transformed plants may be selfed to give homozygous transformed plants, and these plants further propagated through classical breeding techniques. A dominant selectable marker (such as npt II) can be associated with the expression cassette to assist in breeding. Seeds may be collected from mature plants of the present invention in accordance with conventional techniques to provide seed that germinates into a plant as described herein.

The present invention is explained in greater detail in the following non-limiting Examples.

All molecular biology protocols and reagents, unless indicated otherwise, are as described in Current Protocols in Molecular Biology, ed. L. M. Albright, D. M. Coen, & A. Varki, John Wiley & Sons, New York, 1995 or Molecular Cloning, A Laboratory Manual, 2nd edition, 1989, by J. Sambrook, E. F. Fritsch, & T. Maniatis; Cold Spring Harbor Laboratory Press, Cold Spring Harbor, N.Y.

### EXAMPLE 1

Plasmid GH 811:sm GFP Reporter Gene Construct

The plasmid GH811 has a soluble modified GFP coding sequence driven by a mas promoter with a FRT site at the 5′ end of the reporter gene.

Synthesis of FRT Sites. Flp Recombinase Target (FRT) sites were synthesized as complementary oligonucleotides and annealed. Two FRT sites were constructed differing only in the restriction endonuclease sites added at either end. The forward (5′-3′) oligonucleotide sequence for the 5° FRT was 5′-FRT: XhoI-5′-FRT-SalI, as follows:

CGACTCTCGAGGAAGTTCCTATTCCGAAGTTCCTATTCTCTAG;

The antisenise oligonucleotide for the 5′FRT sequence for the 5′-FRT: Xhol-5′-FRT-SalI was:

CAGATGTCGACGAAGTTCCTATACTTTCTAGAGAATAGGAAC.

The forward (5′-3′) oligonucleotide sequence for the 3′ FRT was 3′-FRT:BamHI-3′-FRT-Kpnl, as follows:

CGACTGGATCCGAAGTTCCTATTCCGAAGTTCCTATTCTCTAG.

The antisense oligonucleotide sequence for the 3′ FRT was 3′-FRT: BamHI-3′-FRT-KpnI, as follows:

CAGAGGTACCGAAGTTCCTATACTTTCTAGAGAATAGGAAC.

Each set of oligonucleotides was designed to be complimentary one to the other such that upon annealing a double stranded molecule with 5′ overhangs resulted; in the sequences above, the overlap sequences are underlined. Each sticky-ended duplex was filled-in using T-4 DNA polymerase (New England Biolabs) at an 11 degree centigrade reaction temperature to produce fully double stranded DNA. The double stranded DNA fragments were identified and purified using polyacrylamide gel electrophoresis.

Cloning the FRT Sites. The 5′FRT: The FRTs were then cloned directionally using a stepwise procedure which takes advantage of the fact that the FRT sequence has a unique internal Xbal site; the 5′FRT duplex was cut with XhoI+XbaI and ligated into the vector pSL301 (InVitrogen) cut with the same enzymes. The resulting product was cut with XbaI+SalI and the XbaI+SalI fragment of the 5′FRT duplex ligated in, producing pSL301-5′FRT.

The 3′FRT: 3′FRT was cloned by Cutting the 3′FRT duplex with BamHI+XbaI, inserting this into pSL301 similarly cut. The ligation product from this was then cut with XbaI+KpnI and the XbaI+KpnI fragment from 3′FRT inserted. The 3′FRT BamHI-KpnI fragment was gel isolated and inserted into pSL301-5′FRT similarly cut, producing pSL301-5′FRT-multiple cloning site-3′FRT.

The Mannopine Synthase Promoter (mas)—A unidirectional mas promoter was constructed using PCR amplification of pAGM139 (Gerry Hall, Mycogen) as template and the sense strand oligo:

GCGCACGCGTAAGCTTAGATTTTTCAAATCAGTGCGC, which added

Mlu and HinDIII sites 5′, and an antisense strand oligo:

GCGCATGCATTCTAGACGATTTGGTGTATCGAGATTGG,

which added Nsi and XbaI sites 3′. The product resulting from this was cut with Hind lll+XbaI and the resulting gel-purified fragment ligated into pSL301 cut with Hind lll+SpeI. The resulting product retained the Hindlll site but had the SpeI site destroyed. This mas promoter corresponds to the 318 bp piece from the “mas gene described by Ni et al, 1996, Plant Mol. Biol. 30, 77-96.

Plasmid GH 700. The plasmid Omega Gus (Lynn Dickey, NCSU and Gallie et al., Plant Cell 1, 301-311 (1989)) was cut with XbaI and treated with Klenow to blunt these sites. The resulting linear piece was cut with EcoRI, producing a 2 kb fragment. This is ligated into GH355 cut with NruI and EcoRI, producing GH 669, which has the insert <nosP/Frt/omega-gus/nosT>. Plasmid GH669 was cut with HindIlI and SpeI and the ˜350 bp mas promoter PCR fragment cut with HindIII and XbaI ligated in to produce GH700, which has the <masP/Frt/omega-gus/nosT>insert.

Construction of Plasmid GH 811. GH 700 is cut with BamH1 and EcoRI and the larger BamH1-EcoRI fragment gel isolated. The GFP containing plasmid, psmGFP, was obtained from the Ohio State University Arabidopsis Biological Resource Center. This GFP gene is a soluble modified derivative of the GFP. Plasmid psmGFP was cut with BamH1 and EcoR1 which released a 1 kb fragment bearing the smGFP coding sequence and nos terminator. This fragment was ligated into the GH700 BamHI-EcoRl backbone fragment. This produced the plasmid GH 811, containing the <masP/Frt/smGFP/nosT>DNA.

### EXAMPLE 2

Construction of Plasmid TN 2;

The Double MAR-Fluorescent Reporter Gene Construct

The plasmid TN 2 carries a soluble modified (sm) GFP reporter gene driven by a mas promoter with an FRT site in between. The gene cassette is flanked by double Rb7-MAR sequences in a direct repeat orientation.

The plasmid was constructed with a pKS vector backbone having double Rb7-MAR sequences in a direct repeat orientation derived from plasmid NCGH 5 (Gerry Hall, NCSU) and the smGFP gene cassette from plasmid GH 811. Both plasmids NCGH 5 and GH 811 were cut with Hind III and Eco RI to open up the vector plasmid and release an insert, respectively. The linearized plasmid and insert fragment were gel purified and then ligated to become the plasmid TN 2, which has the <Rb7/mas P/Frt/sm GFP/nos T/Rb7>in the pKS vector backbone.

### EXAMPLE 3

Construction of Plasmid TN1: The Selectable Marker Construct

The plasmid TN 1 has a hygromycin resistance gene, hpt II driven by a mannopine synthase, mas promoter. The construct was made for use in co-bombardment of maize embryos, with the fluorescent screenable plasmid (pTN2). However, selection for hygromycin was not performed for the entire experiment from embryo culture to recovery of T0 plants. The integration of the hygromycin gene in chromosomes of T0 plants and their seed progeny would be useful in subsequent quick screenings of T1 generation.

The unidirectional mas promoter was constructed using PCR amplification of pAGM139 (Gerry Hall, Mycogen) as template and the sense strand oligo:

GCGCACGCGTAAGCTTAGATTTTTCAAATCAGTGCGC, which added Mlu and Hind III sites 5′, and an antisenise strand oligo:

GCGCGTTAACGGATCCCGATTTGGTGTATCGAGATTGG, which added Hpa and BamHI site 3′. The PCR product has 385 bp with Hind III site 5′ and Bam HI site

The hygromycin resistance gene, hpt II derived from plasmid pGA 1434 (George Allen, NCSU), which was digested with Hind III and Bgl II to remove the nos promoter. The resultin linearized promoterless plasmid was gel isolated and ligated with the gel-purified mas promoter fragment to produce pTN 1.

### EXAMPLE 4

Isolation and Culture of Immature Embryos

Seeds of inbred lines M37W, A6 and A188 were obtained from the NCSU maize breeding program headed by Major Goodman. The donor plants were grown to maturity in an environmentally controlled chamber in the NCSU Phytotron. The environmental condition for the entire growth cycle was set at 26° C./22° C. day/night temperature and 27,000 lux light intensity provided by both incandescent bulbs and fluorescence tubes for a 12 hour—light and dark cycle. These plants were either sib or self-pollinated and ear shoots were harvested 9-11 days after pollination for isolation of immature embryos. The isolated embryos measured 0.8 to 1.2 mm in length and varied in development from coleoptilar stage when the apical dome was not covered by leaf primordia to stage 1 embryo when the first leaf primordium had already covered the meristem (A. Van Lammeren, Acta Bot. Neerl. 35, 169-188 (1986)).

The husked ear shoots were harvested and surface sterilized by submerging for 25 minutes in a solution of 50% commercial bleach (containing 4.5% sodium hypochlorite), and 0.1% Tween 20. The ear shoots were dehusked, submerged for 20 minutes in 25% commercial bleach and 0.1% Tweeni 20, and rinsed 3 times with sterile water. The embryos were aseptically isolated by gently squeezing them from the soft kernels using a sterile flat metal spatula under a dissecting microscope. The isolated embryos were plated with scutella surface in contact with the embryo maturation medium containing MS salts (T. Murashlige and F. Skoog, Physiol. Physiol. Plant. 15, 473-497 (1962)) and B5 vitamins (O. Gamborg et al, Exp. Cell Res. 50, 151-158 (1968)), 0.5 mg/l benzyladenin, 1.0 mg/l indole acetic acid, 15% (w/v) sucrose and 0.8% phytagar (GIBCO). Ten to twelve embryos were plated per plate (15×60 mm) with 3 replicates per treatment. One day after plating, embryos were bombarded under the conditions described below.

### EXAMPLE 5

Microprojectile Bombardment of Immature Embryo Meristems

All the consumables used in the bombardment experiments were supplied by Bio-Rad. The DNA microcarriers were prepared as follows: 60 mg of 1.6 μm gold particles were suspended in 1 ml absolute ethanol and vortexed gently. After centrifugation for 10 seconds at 13,000 rpm to remove ethanol, the particles were resuspended and washed twice in 1 ml of sterile deionized water. The gold particle suspension was stored at 4° C. as 50 μl aliquots in 1.5 ml microfuge tubes. Before bombardment, 5 μl of TE buffer containing 2.5 μg DNA mix of the reporter plasmid (TN2) and selectable plasmid (TN1) at 4:1 ratio, were added to 50 μl microcarrier aliquot and pipetted up and down to mix. After mixing, 50 μl CaCl₂ (2.5 M) was added and mixed by pipetting, followed by 20 μl spermidine (0.1M). The mixture was then placed on a vortex platform and agitated at low speed initially then increased slowly over 3-5 minutes; care was taken to keep the mixture from reaching the tube lid. The tubes were centrifuged for 10 seconds at 10,000 rpm. The supernatant was discarded and the DNA-coated gold particles were washed with 250 μl absolute ethanol and resuspended in 65 μl absolute ethanol. Ten μl aliquot was spread on a macrocarrier that was secured onto a macrocarrier holder and used for each bombardment. The delivery of the DNA was done with a PDS-1000 helium gun (Bio-Rad) with rupture disks of 650 psi. The macrocarrier flying distance was 10 mm. Each plate of cultured embryos was bombarded twice with the distance between the stopping screen and embryos of 6 cm in the first shot and 9 cm in the second shot.

### EXAMPLE 6

Screening of Germinating Embryos Expressing GFP

Two weeks after bombardment, the dark-incubated embryos were screened for GFP expression using a fluorescence stereoscopic microscope (Nikon SMZ-U) connected to a spot digital camera. The embryos were viewed with a 2× objective and magnified at 0.75×. The time exposure for all observations was manually set for 60 seconds. Images of individual embryos were recorded for tracking the derivative T0 plants. Only germinating embryos having no accompanying callus were selected for transfer to embryo germination medium containing MS salts, B5 vitamins, 3% sucrose and 0.8% phytagar in capped tubes (40×120 mm). The germinating embryos were kept under dimmed light at 27° C. for 3 weeks before transfer to 10″ pots filled up with a standard mix and grown to maturity in the Phytotron.

### EXAMPLE 7

DNA Extraction from Maize Leaf Tissues

Four inch-segments of flag leaf blades were collected and snap frozen in liquid Nitrogen. The frozen leaf tissues were ground in liquid Nitrogen with a mortar and pestle. The micro-sample size DNA extraction method was adapted from the Molecular Marker Lab, Crop Science Department, NCSU. The DNA extraction buffer contained 0.5 M NaCl, 0.1 M Tris-HCl (pH 8.0), 0.025 M EDTA (pH 8.0), 20% SDS. The buffer was added with 3.8 g/l sodium bisulfite, adjusted to pH to 7.8-8.0 with 10 N NaOH and heated to 65° C. before use. In a 2.0 ml microfuge tube, about **mg ground tissue were mixed with 600 μl DNA extraction buffer and heated at 65° C. for 30-40 minutes with frequent mixing by inverting the tubes 2-3 times every 10 minutes. The mixture was cooled to room temperature before adding 600 μl 24:1 chloroform: isoamyl alcohol and mixed well by inverting the tube several times. After centrifugation at 10,000 rpm in a microcentrifuge for 10 minutes, the supernatant was removed by pipetting and transferred to a clean 1.5 ml microfuge tube. The tube was then filled up to the rim with 95% ethanol and incubated at −20° C. for at least one hour to precipitate the DNA. The DNA was collected by centrifugation at 10,000 rpm for 5 minutes and washed with 300 μl of 70% alcohol. The DNA pellet was air dried, resuspended in 150 μl TE and kept at 4° C. overnight to completely dissolve the DNA. The DNA was then treated with 5 μl of RNase A (10 mg/ml), incubated at room temperature for 30 minutes and centrifuged at 8,000 rpm for 5 minutes. The top 140 μl of the DNA solution was removed with a pipette. The DNA was quantified using a Hoefer TKO 100 fluorometer.

### EXAMPLE 8

PCR Analysis of T0 Plants

The polymerase chain reaction (PCR) conditions and cycling profiles were adapted from the Simple Sequence Repeats (SSR) Methods from the Maize Genome Database with slight modifications. The reaction was set up as follows:

PCR buffer, 1×

Magnesium chloride, 1.0 mM

dATP, dCTP, dGTP, dTTP, 100 μM each

Forward primer, MasSense (25 mer), 5 μM with the following sequence:

5′-GGT CGT TTA TTT CGG CGT GTA GGA C-3′

Reverse primer, smGFP(130)antC(25 mer), 5 μM with the following sequence:

5′-GCA TCA CCT TCA CCC TCT CCA CTG A-3′

Taq polymerase, 1 unit

Non-acetylated BSA, 15 μg

Maize genomic DNA, 75 ng

Sterile DI water to bring final volume to 15 μl.

All thermocycling procedures were performed in 0.5 ml microfuge tubes with an oil overlay using MJ Research PT100 thermocycler. The cycling profile included:

heating at 95° C. for 5 minutes, which was followed by

denaturing 94° C. 1 minute

annealing 65° C. 1 minute

extension 72° C. 2 minutes

for two cycles and then a one-degree decrement for the annealing temperature, each repeated once, until the temperature is 55° C. The regime was then

| | |
|---|---|
| 94° C. | 1 minute |
| 55° C. | 1 minute |
| 72° C. | 2 minutes |

repeated for a total of 40 cycles. A soak cycle at 4° C. was included at the end of the reactions.

A The PCR products amplified from the genomic DNA of T0 plants were analyzed by gel electrophoresis. The amplified DNA solution pipetted carefully to avoid drawing up the overlaid oil and transferred to a clean tube. It was then added with 3 μl of the loading dye and loaded in an ethidium bromide gel [2% agarose in TAE (Tris/Acetic acid/EDTA) buffer]. The gel was run for 1-2 hours at 100 volts in TAE buffer. Samples included a positive control that was the PCR product amplified from the genomic DNA of a GFP-expressing NT1 cell line and confirmed PCR-positive using the same set of primers as above. A 100 bp DNA ladder was used as marker showing a correct band of about 500 bp size from PCR-positive samples.

### EXAMPLE 9

Analysis of Plants

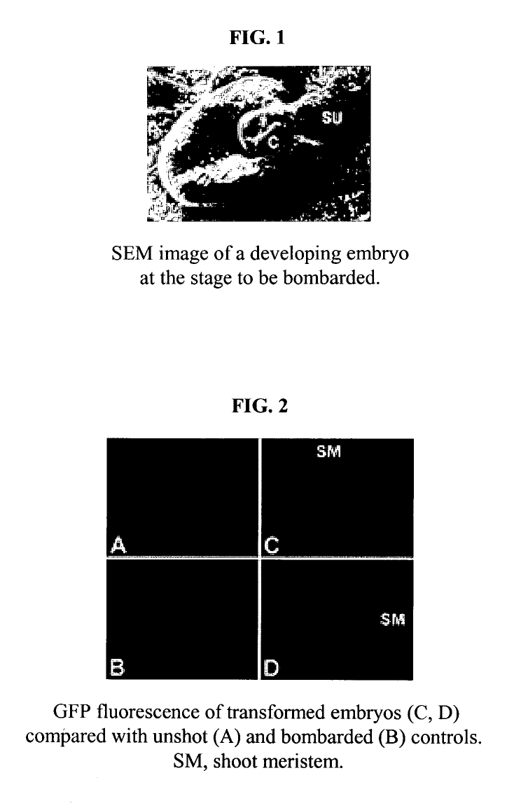

In summary, procedures have been developed that allow one to obtain a high frequency of expression at the shoot apex of immature embryos (FIG. 1). DNA was introduced by bombardment of excised immature embyros from several different inbred lines. Thus far, only a mas; GFP construct developed for dicots has been used. Certain maize lines (e.g., M37W) showed low autofluorescence, and in these cases we were able to screen embyros with a dissecting microscope equipped with fluorescence optics (eg, FIG. 2). This expression is developmentally stable, not transient.

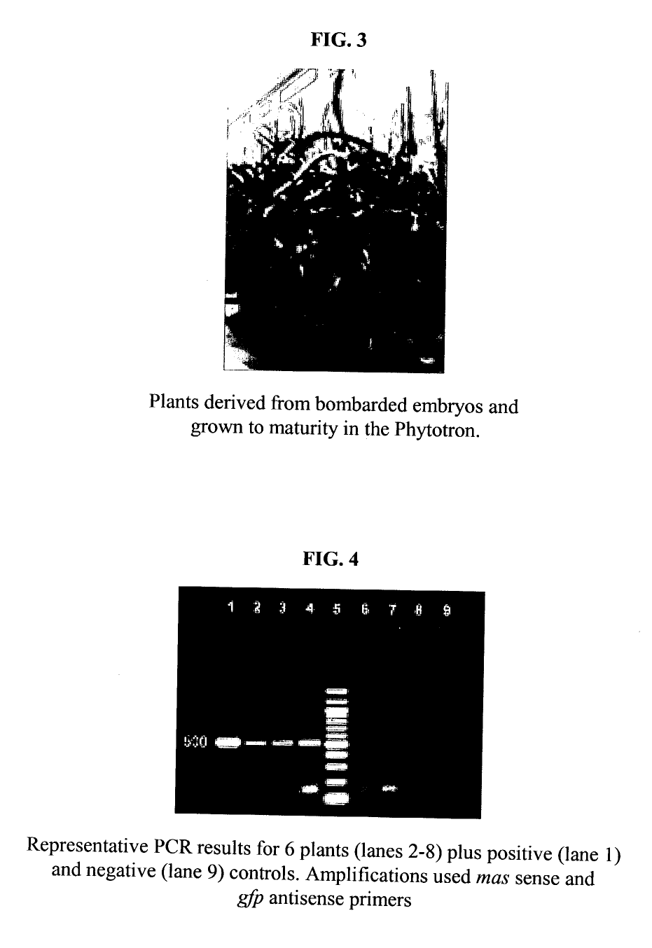

Plantlets derived from in vitro germination of these embryos are vigorous and highly fertile (FIG. 3), perhaps because we have bypassed the usual regeneration and drug selection steps. In about 15%, we obtain positive signals by PCR analysis of DNA extracted from flag leaf tissue (FIG. 4). Because such plants are chimeric, sampling only a portion of a leaf will greatly underestimate the frequency with which transgenes are stably incorporated into cell lineages. Thus we believe we can produce large numbers of vigorous, phenotypically normal, fertile plantlets carrying transgenic sectors.

### EXAMPLE 10

Construction of Plasmid pTN5: The Double MAR GUS Reporter Gene Construct

The plasmid pTN 5 carries a coding sequence for β-glucuronidase (uid A) gene driven by a 2.9 kb fragment that includes the ubiquitin promoter, the 5′-untranslated exon and the first intron of the maize ubiquitin (Ubi-1) gene, and a nos terminator. The gene cassette is flanked by double Rb7-MAR sequences in a direct repeat orientation.

The plasmid was constructed with a pKS vector backbone having double Rb7-MAR sequences in a direct repeat orientation flanking a gene cassette that includes 35S CaMV promoter, uid A coding sequence and nos terminator (pNCGH 11; Gerry Hall, NCSU). The fragment containing the maize ubiquitin promoter with its first exon and first intron was isolated from plasmid pAHC17 (Chistensen and Quail, Transgenic Research 5, 213-218 (1996)). The plasmid pNCGH 11 was cut with HindII and BamH1 to remove the 35S CaMV promoter and open up the backbone. Likewise, the plasmid pAHC 17 was cut with HindIII and BamH1 to release the promoter fragment with the first exon and intron. The linearized plasmid and insert fragment were gel purified and then ligated to become the plasmid TN5, which has the insert <Rb7/ubiP/I/uidA/nosT/Rb7>in the pKS vector backbone.

### EXAMPLE 11

Transformation of Meristems in Immature Embryos with GUS Constructs by Microprojectile Bombardment Immature embryos of inbred line M37W were isolated from ears harvested 10-11 days after pollination. The isolated embryos measured 0.9 to 1.1 mm in length. The donor plants were grown to maturity in an environmentally controlled chamber in the NCSU Phytotron. The environmental condition for the entire growth cycle was set at 26° C./22° C. day/night temperature and 27,000 lux light intensity provide by both incandescent bulbs and fluorescence tubes for a 16 hour-light and dark cycle. These plants were either sib or self-pollinated.

The procedures of surface sterilization of the ear shoots, isolation and plating of immature embryos were same as that under Example 4. The embryo maturation medium stated under Example 4 was used with a reduced amount of sucrose to 12% instead of 15%.

The microprojectile bombardment protocol listed in Example 5 was used with 0.6 μm microcarriers. GUS reporter plasmids having either no Rb7 MAR (pAGM606—from Mycogen) or double Rb7 MAR (pTN5—Example 10) were mixed with the was used for each shot.

### EXAMPLE 12

Evaluation on Transient and Stable Expression of GUS

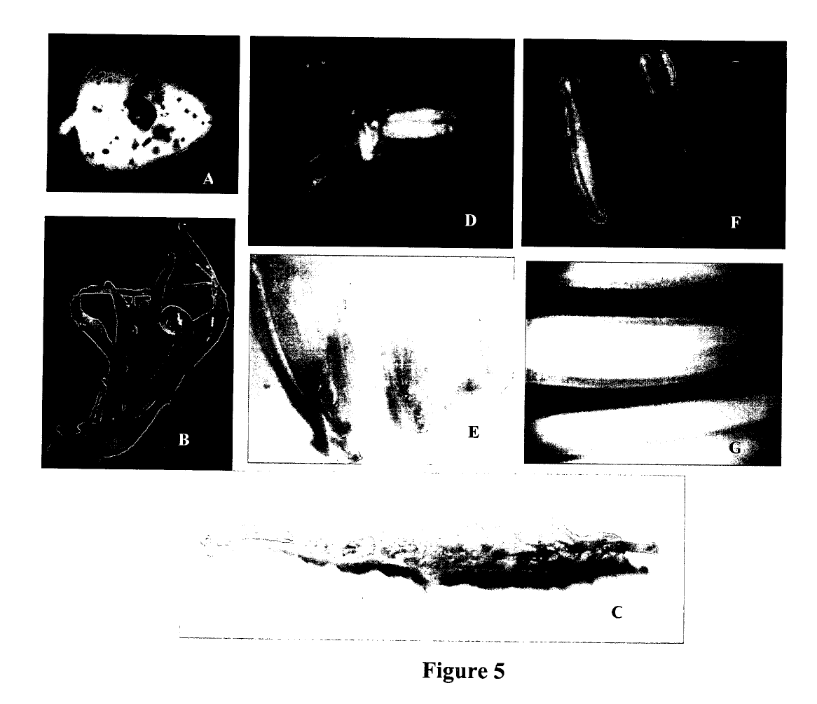

Three days after bombardment, the transient expression of β-glucuronidase oil bombarded embryos was assayed histochemically following the GUS staining protocol of R. Jefferson et al., EMBO J 6, 3901-3907 (1987)). Numerous dark blue spots were observed on the embryonic axis especially on the coleoptilar ring enclosing the shoot apical meristem (FIG. 5A).

Stable expression of the transgene was assayed histochemically at 4 weeks after bombardment and before the emergence of tassels. Blue sectors on young seedlings were recorded (FIG. 5B). When we stained young tassels (0.5-3 cm length), we found GUS activity in 1 out of 8 tassels from plants bombarded with the MAR construct (TN5). No expression was detected in plants bombarded with the non-MAR construct (pAGM606). At a later stage of development but still before emergence; tassels measuring up to 13 cm in length were sampled for GUS histochemical assay. We obtained 6 GUS-positive tassels with blue sectors (FIG. 5C), out of a total of 19 collected samples from plants bombarded with the MAR construct, and 3 positives out of 15 for the non-MAR construct. GUS expressing florets (FIG. 5D) were observed with pairs of chimeric anther for the transgene (FIG. 5E). Microscopic observation of one GUS expressing tassel revealed numerous blue-stained anthers (Figure F, G) at about 30% of the total counted anthers. At the development stage before emergence of the tassel, microspores are not fully developed and transgenic microspores were not conspicuously visualized.

The foregoing is illustrative of the present invention, and is not to be construed as limiting thereof. The invention is defined by the following claims, with equivalents of the claims to be included therein.

1. A method for introducing a heterologous nucleic acid of interest into a plant to thereby produce a recombinant plant, said method comprising the steps of:
(a) providing a recombinant nucleic acid of interest, said recombinant nucleic acid comprising said heterologous nucleic acid of interest and (i) a matrix attachment region (MAR) positioned 5′ to said heterologous nucleic acid of interest, (ii) a MAR positioned 3′ to said heterologous nucleic acid of interest, or (iii) a MAR positioned 5′ to said heterologous nucleic acid of interest and a MAR positioned 3′ to said heterologous nucleic acid of interest;
(b) providing meristem tissue of said plant of interest;
(c) introducing said recombinant nucleic acid of interest into said meristem tissue; and then
(d) directly propagating a recombinant plant from said meristem tissue.

2. The method according to claim 1, wherein said plant is a dicot.

3. The method according to claim 1, wherein said plant is a monocot.

4. The method according to claim 1, wherein said plant is a grass species.

5. The method according to claim 1, wherein said plant is selected from the group consisting of maize, wheat, oats, rye, barley, sorghum, and rice.

6. The method according to claim 1, wherein said plant is a maize plant.

7. The method according to claim 1, wherein said plant is a hybrid plant.

8. The method according to claim 1, wherein said plant is an inbred plant.

9. The method according to claim 1, wherein said introducing step is carried out by direct DNA delivery.

10. The method according to claim 1, wherein said introducing step is carried out by microparticle bombardment.

11. The method according to claim 1, wherein said introducing step is carried out by Agrobacterium-medicated transformation.

12. The method according to claim 1, wherein said meristem comprises post-germination plant meristem.

13. The method according to claim 1, wherein said meristem comprises adult plant meristem.

14. The method according to claim 1, wherein said meristem comprises seedling meristem.

15. The method according to claim 1, wherein said meristem comprises embryo meristem, and wherein said propagating step is carried out by in vitro germination.

16. The method according to claim 1, wherein said plant is a grass species, said meristem tissue comprises an embryo apical meristem taken at a time of development between the formation of the apical meristem and before the apical meristem is occluded by coleoptile tissue, and said propagating step is carried out by in vitro germination.

17. The method according to claim 1, wherein said plant is a maize plant, said meristem tissue comprises embryo apical meristem taken at a time of development from 7 to 14 days after pollination, and said propagating step is carried out by in vitro germination.

18. The method according to claim 1, further comprising the step of sexually propagating said plant to produce a plant that is hemizygous or homozygous for said heterologous DNA of interest.

19. The method according to claim 1, wherein said heterologous nucleic acid of interest comprises a structural gene operably associated with a promoter active in cells of said plant, and wherein cells of said plant exhibit increased expression of said structural gene as compared to cells of the same plant that do not contain said heterologous nucleic acid of interest.

20. The method according to claim 1, wherein said heterologous nucleic acid of interest comprises DNA.

21. The method according to claim 1, wherein said introducing step is carried out by introducing said heterologous nucleic acid of interest into meristem L2 cells.

22. The method according to claim 1, wherein said propagating step comprises propagating a mature recombinant plant from said meristem tissue which carries said heterologous nucleic acid in gametophyte cells thereof.

23. The method of claim 22, wherein said gametophyte cells are male gametophyte cells.

24. The method of claim 22, wherein said gametophyte cells are female gametophyte cells.

25. A plant produced by the method of claim 1.

26. Seed, pollen or propagules collected from a plant of claim 21 and which carry said heterologous nucleic acid.
